(19) United States
(12) Patent Application Publication
(10) Pub. No.: US 2026/0027309 A1
(43) Pub. Date: Jan. 29, 2026

(54) HIGH FREQUENCY DROPLET DELIVERY SYSTEM AND METHOD

(71) Applicant: PNEUMA RESPIRATORY, INC., Boone, NC (US)

(72) Inventors: Judson Sidney Clements, Boone, NC (US); Chao-Ping Lee, Boone, NC (US); Michael Scoggin, Boone, NC (US); Jeffrey Miller, Boone, NC (US)

(73) Assignee: PNEUMA RESPIRATORY, INC., Boone, NC (US)

(21) Appl. No.: 18/997,377

(22) PCT Filed: Jul. 18, 2023
(86) PCT No.: PCT/US2023/028050

§ 371 Date: Jan. 21, 2025

Related U.S. Application Data
(60) Provisional application No. 63/390,170, filed on Jul. 18, 2022.

(51) Int. Cl. A61M 11/00; A61K 9/00; B05B 17/06

(52) U.S. Cl. CPC A61M 11/005; A61K 9/0078; B05B 17/06; A61M 2205/0294

(57) ABSTRACT

A method of aerosol generation includes operating an electronic transducer at a second higher frequency than a first lower frequency with a same fluid composition and same ejector mechanism mesh with a predetermined hole size and generating smaller droplets at the second higher frequency.

### FIELD

The present disclosure relates generally to aerosol generation devices that include a vibrating transducer, such as piezoelectric transducer. Examples include droplet delivery devices that deliver fluids that are inhaled into the mouth, throat, nose, and/or lungs.

### BACKGROUND

Aerosol generation devices may include a vibrating transducer, such as a piezoelectric transducer, to create aerosolized droplets for a variety of applications. Some aerosol generation devices include droplet delivery systems directed to both therapeutic and non-therapeutic uses. Current droplet delivery systems include a variety of inhaler type systems. Some examples are metered dose inhalers (MDI), pressurized metered dose inhalers (p-MDI), pneumatic devices, and ultrasonic-driven devices. Such droplet delivery systems are directed to both therapeutic and non-therapeutic uses and may include mouthpieces and nosepieces to provide for inhalation of the fluid droplets.

### BRIEF DESCRIPTION OF THE DRAWINGS

The novel features of the disclosure are set forth with particularity in the appended claims. A better understanding of the features and advantages of the present inventive concept will be obtained by reference to the following detailed description that sets forth illustrative examples, in which the principles of the disclosure are utilized, and the accompanying drawings of which:

FIG. 1 illustrates generating a droplet using a lower frequency and a higher frequency for a given mesh size according to an embodiment of the disclosure.

FIG. 2 is a cross-sectional view of major components of a droplet delivery device in accordance with an embodiment of the disclosure.

FIG. 3 illustrates a cross-sectional view of a vibrating member enclosure of a droplet delivery device utilizing membrane-driven aerosolization in accordance with one embodiment of the disclosure.

FIG. 4A illustrates a side plan view of an exemplary aperture plate and annulus ring, in accordance with an embodiment of the disclosure.

FIG. 4B illustrates a top plan view of an exemplary aperture plate and annulus ring, in accordance with an embodiment of the disclosure.

FIG. 4C illustrates a cross-section of an exemplary aperture plate and annulus ring configuration, in accordance with an embodiment of the disclosure.

### DETAILED DESCRIPTION

As used herein, the terms “comprises,” “comprising,” “includes,” “including,” “has,” “having” or any other variation thereof, are intended to cover a non-exclusive inclusion. For example, a process, product, article, or apparatus that comprises a list of elements is not necessarily limited only those elements but can include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term substantially, as used herein, is defined to be essentially conforming to the particular dimension, shape or other word that substantially modifies, such that the component need not be exact. For example, substantially cylindrical means that the object resembles a cylinder, but can have one or more deviations from a true cylinder.

The term “coupled” is defined as connected, whether directly or indirectly through intervening components, and is not necessarily limited to physical connections. The connection can be such that the objects are permanently connected or releasably connected. The term “comprising” means “including, but not necessarily limited to”; it specifically indicates open-ended inclusion or membership in a so-described combination, group, series and the like.

Droplet delivery devices include an ejector mechanism with a mesh, aperture plates and like substrates having desirably sized holes and producing desirable surface contact angle that creates droplets from liquid passing through the mesh when a powered transducer acts on the liquid and ejector mechanism. In some devices a membrane may be oscillated by a powered transducer to push the liquid through the mesh and create droplets (“push mode”), while in other devices a transducer can be coupled directly to oscillate the mesh to create droplets. Examples of devices including such ejector mechanisms with substrates having apertures are described in U.S. Patent Application Pub. No. US2022/0401661 entitled “DELIVERY DEVICE WITH PUSH EJECTION” published Dec. 22, 2022, International Publication Number WO 2020/264501 entitled “DELIVERY OF SMALL DROPLETS TO THE RESPIRATORY SYSTEM VIA ELECTRONIC BREATH ACTUATED DROPLET DELIVERY DEVICE” published Dec. 30, 2020, and International Publication Number WO 2020/227717 entitled “ULTRASONIC BREATH ACTUATED RESPIRATORY DROPLET DELIVERY DEVICE AND METHODS OF USE” published Nov. 12, 2020, all of which are herein incorporated by reference in their entirety, including incorporation of such publications and patent applications as are cited and incorporated by reference or relied upon in the referenced disclosures.

The present technology implements a mesh that vibrates to generate droplets. The configuration of the mesh and other components can vary, but at least one example is presented herein. The present technology implements a high frequency vibration of the mesh to generate smaller droplet sizes. The present technology achieves the high frequency vibration that results in the droplets for a given mesh hole size being smaller. A higher frequency piezoelectric device allows the mesh to vibrate at a higher frequency. This results in the less liquid passing through each hole in the mesh per vibration because the liquid has less time to flow through the holes. If a low frequency is used there is more time for the liquid to continuously come out of the hole. The liquid jets out of the hole in one piece and then turns into a droplet. At a higher frequency, less liquid exits the hole before the mesh reverses direction and thereby cuts the liquid flow off. Therefore, the droplet that is generated is smaller than the droplet generated by the lower frequency piezoelectric device. The piezoelectric device operates by receiving a signal that in turn causes it to vibrate at one or more frequencies. As described herein, the piezoelectric is operable to vibrate at high frequencies thereby creating smaller droplet sizes.

Using a higher frequency piezoelectric device allows for the mesh hole size to be increased for the same size droplet. This can have at least two benefits. The first is that the mesh can be more easily be produced and controlled in mesh hole size. Second, the larger mesh hole size can reduce the shearing involved in generating the droplet. However, there is a maximum frequency for a given mesh hole size as the higher the frequency there is a point at which the liquid will not make it through the mesh. For example, with past technology the goal of achieving a 1 micrometer droplet size requires a 1.7 micrometer mesh hole size. This is one of the smaller mesh hole sizes that are possible with existing technology. The present technology can use mesh hole sizes that are on the order of 2 micrometer and still achieve a 1 micrometer droplet size. Thus, it is possible to use larger mesh hole sizes and still achieve the same droplet size. It is also important to consider the liquid viscosity and surface tension in selecting the mesh hole size and frequency as these two additional factors also influence how large the droplet size will be.

The present technology can be implemented with a small mesh hole size to decrease the droplet size. In those situations in which sheer stress on the liquid is a factor, the mesh hole size can be increased and the frequency increased to generate a small droplet.

In another example, the mesh hole size can be as described above, but a different driving mechanism can be used to generate the jet. The piezoelectric transducer can vibrate a horn (also called a vibrating member) instead of the mesh as described above. The horn can then cause the fluid to be expelled through the mesh resulting in a jet of fluid.

The operation of the other example can be explained such that as the horn pulls backward, the horn causes a pause in the ejection, even if the mesh is still vibrating. The analogous behavior is similar to a pumping behavior. As the horn pushes forward, the horn is pressing the liquid against the mesh. As the horn pulls back, it is pulling liquid in between the horn and the mesh.

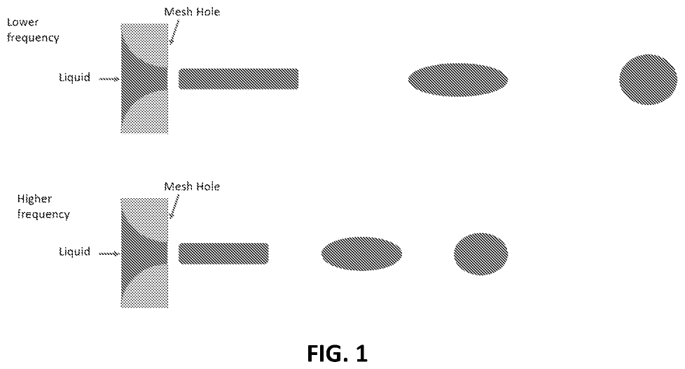

FIG. 1 illustrates generating a droplet using a lower frequency and a higher frequency for a give mesh hole size. As illustrated, the lower frequency generates a larger droplet as compared to the higher frequency. Initially, the liquid exits the mesh in the form of a jet (slug) and then changes into a droplet due to the surface tension of the liquid.

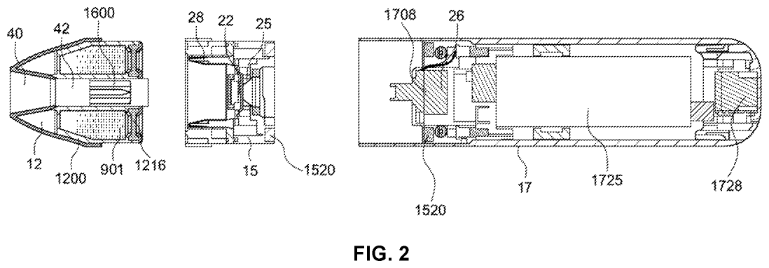

An example device according to the present technology can be described as follows and illustrated in FIG. 2. A droplet delivery device having a membrane that cooperates with a mesh further includes a PZT-based ultrasonic transducer coupled to a vibrating member having a tip portion made of at least one of Grade 5 titanium alloy, Grade 23 titanium alloy, and about 99% or higher purity titanium. In certain embodiments, the vibrating member's tip includes a sputtered on outer layer of and about 99% or higher purity titanium providing a smooth tip surface configured to contact an underlying bottom surface of the membrane that is opposite an exterior top surface of the membrane positioned nearest the mesh so as to help reduce wear of the membrane and increase the longevity and operation consistency of the membrane (and also possibly vibrating member's tip portion). The transducer can be configured to have the desired higher frequency as described above. A vibrating member 1708 and transducer 26 that work in conjunction with a membrane 25 and mesh 22 to aerosolize fluid 901, which is held in a reservoir 1200 and supplied to the mesh 22 using various methods (e.g., wick material, hydrophilic coatings, capillary action, etc.). Preferably the vibrating member is coupled to the transducer, such as by bonding (e.g. adhesives and the like), welding, gluing, physical connections (e.g. brackets and other mechanical connectors), and the like. The transducer and vibrating member interact with the membrane to push fluid through the mesh. As illustrated and described in various embodiments, the membrane may in some cases contact the mesh while also “pushing” fluid through holes in the mesh, and may in other cases be separated without contacting the mesh to push liquid through holes in the mesh. The transducer may comprise one or more of a variety of materials (e.g., PZT, etc.). In certain embodiments the transducer is made of lead-free piezoelectric materials to avoid creation of unwanted or toxic materials in a droplet delivery device intended for human inhalation. The vibrating member may be made of one or more of a variety of different materials (e.g., titanium, etc.). The mesh may be one or more of a variety of materials (e.g., palladium nickel, polyimide, etc.). After the fluid is pushed through the mesh, a droplet spray is formed and ejected through a mouthpiece port, carried by entrained air.

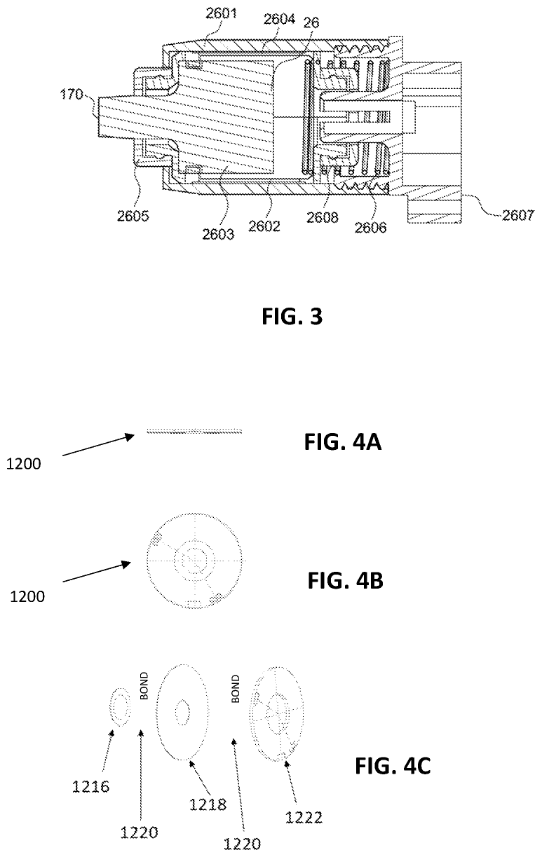

As shown in FIG. 3, the main body of a second example contains the vibrating member and transducer assembly 2603. The vibrating member and transducer assembly 2603 is encased by a vibrating member front cover 2602 and vibrating member rear cover 2604. The covers 2602, 2604 are held together by circular caps called the front and rear vibrating member cover holders 2605, 2608. The encased vibrating member is then put into the vibrating member enclosure 2601, followed by the vibrating member assembly spring 2606, and finally seated into the vibrating member device bracket 2607. The vibrating member enclosure allows the spring to press the vibrating member and transducer assembly to the membrane.

FIGS. 4A-4C illustrate an aperture plate assembly 1200 that includes an aperture plate 1216 (e.g., palladium-nickel) supported by a stainless-steel annulus 1218. The aperture plate is welded or bonded 1220 to the stainless-steel annulus 1218, thereby together allowing a thicker support material which is much less expensive than aperture plate material alone e.g., palladium-nickel. The stainless steel and aperture plate are bonded 1220 to the piezo-electric material 1222 wherein all the components form the aperture plate assembly 1200.

One aspect of the invention provides method of aerosol generation comprising operating an electronic transducer to vibrate at a first frequency; coupling a mesh having a predetermined hole size to the electronic transducer; providing a fluid composition to the mesh while the transducer is vibrating at the first frequency; operating the electronic transducer at a second frequency that is higher than the first frequency; providing the same fluid composition to the mesh while the transducer is vibrating at the second frequency; and generating smaller droplets through the mesh at the second frequency than at the first frequency while maintaining the predetermined hole size.

In another aspect of the invention, a membrane is coupled between a vibrating member coupled to the electronic transducer and the mesh.

In another aspect, an inventive method creates inhalable droplets that may be therapeutic, including medicinal, biological and pharmaceutical treatments, or non-therapeutic, such as simulating smoking, providing flavoring and like activities.

Another aspect of the invention includes a method of aerosol generation comprising: operating an electronic transducer to vibrate at a first frequency; coupling a mesh having a predetermined hole size to the electronic transducer, wherein the hole size is larger than the desired size of droplets to be produced by the mesh; providing a fluid composition to the mesh while the transducer is vibrating at the first frequency, wherein the fluid contains at least one of liposomes, other vesicles and cells; operating the electronic transducer at a second frequency that is higher than the first frequency; providing the same fluid composition to the mesh while the transducer is vibrating at the second frequency; and generating droplets experiencing lower shear in at least a portion of the fluid composition passing through mesh holes located within a central region of the mesh.

In an embodiment of such other aspect of the invention, a membrane is coupled between a vibrating member coupled to the electronic transducer and the mesh.

While the invention has been described with reference to exemplary embodiments, it will be understood by those skilled m the art that various changes may be made, and equivalents may be substituted for elements thereof without departing from the scope of the invention. In addition, many modifications may be made to adapt a particular situation or material to the teachings without departing from the essential scope thereof. Therefore, it is intended that the invention is not limited to the particular embodiment disclosed as the best mode contemplated for carrying out this invention, but that the invention will include all embodiments falling within the scope of the appended claims.

What is claimed is:

1. A method of aerosol generation comprising:
operating an electronic transducer to vibrate at a first frequency;
coupling a mesh having a predetermined hole size to the electronic transducer;
providing a fluid composition to the mesh while the transducer is vibrating at the first frequency;
operating the electronic transducer at a second frequency that is higher than the first frequency;
providing the same fluid composition to the mesh while the transducer is vibrating at the second frequency; and
generating smaller droplets through the mesh at the second frequency than at the first frequency while maintaining the predetermined hole size.

2. The method of claim 1 wherein a membrane is coupled between a vibrating member coupled to the electronic transducer and the mesh.

3. The method of claim 2 further comprising producing inhalable droplets.

4. The method of claim 3 wherein the inhalable droplets are therapeutic.

5. The method of claim 3 wherein the inhalable droplets are non-therapeutic.

6. The method of claim 1 further comprising producing inhalable droplets.

7. The method of claim 6 wherein the inhalable droplets are therapeutic.

8. The method of claim 6 wherein the inhalable droplets are non-therapeutic.

9. A method of aerosol generation comprising:
operating an electronic transducer to vibrate at a first frequency;
coupling a mesh having a predetermined hole size to the electronic transducer, wherein the hole size is larger than the desired size of droplets to be produced by the mesh;
providing a fluid composition to the mesh while the transducer is vibrating at the first frequency, wherein the fluid contains at least one of liposomes, other vesicles and cells;
operating the electronic transducer at a second frequency that is higher than the first frequency;
providing the same fluid composition to the mesh while the transducer is vibrating at the second frequency; and
generating droplets experiencing lower shear in at least a portion of the fluid composition passing through mesh holes located within a central region of the mesh.

10. The method of claim 9 wherein a membrane is coupled between a vibrating member coupled to the electronic transducer and the mesh.

11. The method of claim 10 further comprising producing inhalable droplets.

12. The method of claim 11 wherein the inhalable droplets are therapeutic.

13. The method of claim 11 wherein the inhalable droplets are non-therapeutic.

14. The method of claim 9 further comprising producing inhalable droplets.

15. The method of claim 14 wherein the inhalable droplets are therapeutic.

16. The method of claim 14 wherein the inhalable droplets are non-therapeutic.
